(19) United States
(12) Patent Application Publication
(10) Pub. No.: US 2023/0310688 A1
(43) Pub. Date: Oct. 5, 2023

(54) SENSOR SYSTEMS AND METHODS

(71) Applicant: Soter Technologies, LLC, Ronkonkoma, NY (US)

(72) Inventor: Cary Chu, Mount Sinai, NY (US)

(21) Appl. No.: 18/011,880

(22) PCT Filed: Jun. 30, 2021
(86) PCT No.: PCT/US2021/039879

§ 371 Date: Dec. 21, 2022

Related U.S. Application Data
(60) Provisional application No. 63/046,188, filed on Jun. 30, 2020.

(51) Int. Cl. A61L 9/20; F24F 8/22; F24F 8/24; F24F 11/63; A61L 9/14

(52) U.S. Cl. CPC A61L 9/20; F24F 8/22; F24F 8/24; F24F 11/63; A61L 9/14; F24F 2120/14

(57) ABSTRACT

A sensor system for identifying potential bullying and presence of a moving object at a site includes a sound sensor configured to sense sounds at the site, a motion sensor configured to detect a motion at the site, a sterilizer configured to sterilize air in the site, and a controller configured to identify potential bullying based on the sensed sound, identify presence of the moving object at the site based on the sensed sound or detection of the motion, and supply power to the sterilizer based on the identification.

### CROSS-REFERENCE TO RELATED APPLICATIONS

The present application claims the benefit of and priority to U.S. Provisional Application Ser. No. 63/046,188, filed on Jun. 30, 2020, the entire content of which being incorporated herein by reference.

### BACKGROUND

### Technical Field

The present disclosure relates to a system and method for identifying vaping, potential bullying, and detecting presence of a moving object at an enclosed area. More particularly, the present disclosure relates to a sensor system which includes an air quality sensor for detecting air quality, a sound sensor for detecting sounds, and the sound sensor and/or a motion sensor for detecting presence of a moving object for sanitization. The content of the two-page document (the “Exhibit”) submitted with this present disclosure, as an exhibit, and titled “FLYSENSE” is incorporated herein by reference in its entirety.

### Background of Related Art

Potential bullying, airborne diseases, and vaping have been serious problems in enclosed areas of academic/business environments due to hazardous/harmful impacts on people. Various methods and systems have been developed to identify or prevent potential bullying, vaping, and airborne diseases in enclosed areas, such as classrooms, restrooms, bathrooms, storage rooms, hospital rooms, or other kinds of enclosed areas in a school, hospital, warehouse, cafeteria, offices, financial institutes, governmental buildings, or any business entities. For example, potential bullying and vaping/smoking can be identified by camera surveillance. However, such camera surveillance systems have not been used in private areas such as restrooms, bathrooms, shower rooms, or hospital rooms because privacy has more weights than identification of potential bullying and vaping/smoking.

Potential bullying can be detected by a sound sensor at the site. However, there are many other sounds (e.g., flushing, conversions, cleaning, gaming, or sounds from outside) preventing from identification of potential bullying. Thus, further developments are needed in identification of potential bullying at enclosed areas.

Due to the recent outbreak of COVID-19, heightened alerts have been received on airborne diseases, which are to be transmitted through air. Thus, sterilization of air inside of an enclosed area is more important than ever before. Several means for sterilization have been used to sterilize the air. However, due to harmful effects caused by sterilization the air, the sterilization is to be performed when there is no person in the enclosed area.

Accordingly, effective identification of potential bullying, detection of presence of a moving object, and vaping/smoking is in dire need in enclosed areas for safety and public health purposes.

### SUMMARY

The present disclosure is directed to a sensor system for identifying potential bullying and presence of a moving object at a site. The sensor system includes a sound sensor configured to sense sounds at the site, a motion sensor configured to detect a motion at the site, a sterilizer configured to sterilize air in the site, and a controller configured to identify potential bullying based on the sensed sound, identify presence of the moving object at the site based on the sensed sound or detection of the motion, and supply power to the sterilizer based on the identification.

According to aspects of the present disclosure, the potential bullying is identified when a level of the sensed sound is greater than or equal to a first threshold, and the presence of the moving object is identified when the level of the sensed sound is greater than or equal to a second threshold, which is less than the first threshold.

According to further aspects of the present disclosure, the sterilizer is an ultraviolet (UV) lamp. The UV lamp emits light having a frequency from about 100 nm to about 280 nm. The UV lamp is turned on for a first predetermined period when the presence of the moving object is identified.

According to further aspects of the present disclosure, the controller is further configured to track a total period during which the UV lamp is powered on. The total period is controlled to be less than or equal to a total predetermined period per unit time based on production of ozone by the UV lamp. The total predetermined period is determined based on an area of the site. The controller is further configured to power off the UV lamp for at least a second predetermined period after the UV lamp is turned on for the first predetermined period.

According to further aspects of the present disclosure, the sterilizer includes hydrogen peroxide. The hydrogen peroxide is sprayed when the sterilizer is powered on.

According to still further aspects of the present disclosure, the sensor system is positioned at an entrance of air inflow of a ventilation system.

The present disclosure is also directed to a method for identifying potential bullying and presence of a moving object at a site. The method includes sensing sound by the sound sensor, detecting a motion of the moving object, identifying potential bullying from the sensed sound, identifying presence of a moving object based on the sensed sound or detection of the motion, and supplying power to a sterilizer after the presence of the moving object is identified.

According to aspects of the present disclosure, the potential bullying is identified when a level of the sensed sound is greater than or equal to a first threshold, and the presence of the moving object is identified when the level of the sensed sound is greater than or equal to a second threshold, which is less than the first threshold. Supplying the power includes turning on a ultraviolet (UV) lamp for a first predetermined period when the presence of the moving object is identified.

According to aspects of the present disclosure, the method further includes tracking a total period during which the UV lamp is powered on. The method also includes controlling the total period to be less than or equal to a total predetermined period per unit time based on production of ozone by the UV lamp.

According to further aspects of the present disclosure, the total predetermined period is determined based on an area of the site. The method further includes discontinuing supply of the power to the UV lamp for at least a second predetermined period after the UV lamp is supplied with power for the first predetermined period.

The present disclosure is further directed to a non-transitory computer-readable storage medium including instructions that, when executed by a computer, cause the computer to perform a method for identifying potential bullying and presence of a moving object at a site. The method includes sensing sound by a sound sensor, detecting a motion of the moving object by a motion sensor, identifying potential bullying from the sensed sound, identifying presence of a moving object based on the sensed sound or detection of the motion, and supplying power to a sterilizer after the presence of the moving object is identified.

The present disclosure is still further directed to a sensor system for identifying vaping and detecting presence of a moving object. The sensor system includes an air sensor configured to sense volatile organic compounds (VOCs) in air at a site, a sound sensor configured to sense sounds at the site, a motion sensor configured to detect a motion at the site, a sterilizer configured to sterilize air in the site, and a controller configured to identify vaping based on signature in the sensed VOCs and identify presence of the moving object at the site based on the sensed sound or detection of the motion, and supply power to the sterilizer based on the identification.

According to aspects of the present disclosure, the VOCs can include carbon dioxide and carbon monoxide. The signature is predetermined ranges of predetermined parameters. The presence of the moving object is identified when the level of the sensed sound is greater than or equal to a threshold.

According to further aspects of the present disclosure, the sterilizer is an ultraviolet (UV) lamp. The UV lamp emits light having a frequency from about 100 nm to about 280 nm. The UV lamp is turned on for a first predetermined period when the presence of the moving object is identified.

According to still further aspects of the present disclosure, the controller is further configured to track a total period during which the UV lamp is powered on. The total period is controlled to be less than or equal to a total predetermined period per unit time based on production of ozone by the UV lamp. The total predetermined period is determined based on an area of the site. The controller is further configured to power off the UV lamp for at least a second predetermined period after the UV lamp is turned on for the first predetermined period.

### BRIEF DESCRIPTION OF THE DRAWINGS

A better understanding of the features and advantages of the disclosed technology will be obtained by reference to the following detailed description that sets forth illustrative embodiments, in which the principles of the technology are utilized, and the accompanying drawings of which:

FIGS. 1A and 1B are block diagrams of a sensor system for identifying potential bullying and vaping/smoking and detecting presence of a moving object in accordance with embodiments of the present disclosure;

FIG. 2 is a functional block diagram of the sensor system of FIG. 1 in accordance with embodiments of the present disclosure;

FIG. 3A is a graphical illustration showing detected sound results from the sensors of the sensor system of FIG. 1 in accordance with embodiments of the present disclosure;

FIGS. 3B and 3C are graphical illustration showing history data from the sensors of the sensor system of FIG. 1 in accordance with embodiments of the present disclosure;

FIG. 4 is a flowchart showing a learning mode for the sensors of the sensor system in accordance with embodiments of the present disclosure;

FIG. 5 is a flowchart showing an active mode for the sensor system in accordance with embodiments of the present disclosure;

FIG. 6 is a flowchart showing a method for detecting vaping in accordance with embodiments of the present disclosure;

FIG. 7 is a flowchart showing a method for detecting presence of a moving object with embodiments of the present disclosure; and

FIG. 8 is a functional block diagram of a computing device in accordance with embodiments of the present disclosure.

### DETAILED DESCRIPTION

This disclosure relates to sensor systems for detecting air quality, sound, and motion to identify whether potential bullying and vaping (or other smoking activities) occurs and whether a moving object is present at enclosed areas. When potential bullying and/or vaping are identified, warnings or alerts are transmitted to registered users or clients without providing any indication of warnings to one or more persons who vape or bully at the site. In this way, one or more persons who bully or vape can be properly reported and appropriately supervised later. Further, one or more persons near the vaping or potential bullying can be effectively prevented from further harms.

When a moving object (such as a people or a pet) is not detected, the air in the enclosed area sanitized for a period of time, during which a harmful substance (e.g., ozone) may not be generated at a level so as to minimize damages to the people or pet in the enclosed area.

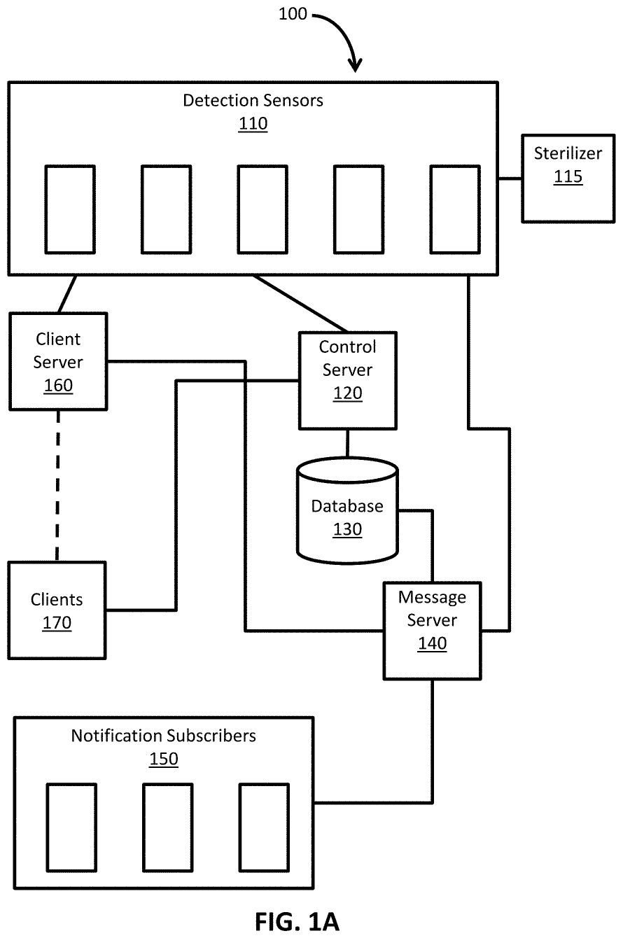

FIG. 1 illustrates a block diagram showing a sensor system 100 according to embodiments of the present disclosure. The sensor system 100 includes a plurality of sensors 110, which detect air quality related to vaping, sound related to noise disturbance, and a motion to detect presence of a moving object at enclosed areas 190. The sensor system 100 is presented in the Exhibit as sanitizing environment with a vape detector. The sensor system 100 further includes a control server 120 for identifying whether or not vaping or potential bullying occurs or a moving object is detected at the enclosed area 190, and a database 130 storing base data for identifying potential bullying and presence of a moving object and history data of detected sounds and air quality at each enclosed area 190.

The sensors 110 may include an air quality sensor configured to sense air quality, a sound sensor configured to sense sound, and a motion sensor configured to sense a motion. The sensors 110 may further include other types of sensors capable of sensing air quality, sound, and motion. The detected air quality may be analyzed by the sensors 110 or the detected air quality may be transmitted to the control server 120 together with the detected sound. The control server 120 may analyze the detected sound based on base data stored at the database 130 and the detected air quality, and determine whether potential bullying and/or vaping occurs at the enclosed areas 190. Further, based on the detected sound or motions, the control server 120 may determine whether or not a moving object is present at the enclosed areas 190.

When a moving object is not present for a predetermined period, a sterilizer 115 may be activated to sterilize the air in the site. Due to generation of potentially harmful substance (such as ozone), the duration of activation of the sterilizer 115 may be limited to a predetermined period, such as 30 minutes, 15 minutes, 10 minutes, etc. according to the dimension of the enclosed area 190. Further, the total amount of activation may also be limited according to the dimension of the enclosed area 190.

The location of the sterilizer 115 may be different from the installation location of the sensor system 110. For example, the sensor system 110 may be installed at the entrance of inflow of air in the ventilation system 180, while the sterilizer 115 may be installed at any place on the ceiling of the enclosed area 190. Further, based on the dimension of the enclosed area 190, one or more sterilizers 115 may be installed in the enclosed area 190.

The sterilizer 150 may be an ultraviolet light (UV) lamp, which emits UV light, whose frequency is from about 100 nm to about 280 nm, to sterilize the air, as shown in the Exhibit. The UV light is very effective in killing germs in the air but at the same time, generates ozone, which is potentially harmful to people and pets. Thus, the activation time of the UV lamp and the total activation of the UV lamp for a unit time (e.g., a day, working hours, business hours, etc.) may be limited to a certain period, which ensures sterilization of airborne germs and protection of health of people and pets.

The base data stored at the database 130 may be location-dependent, meaning that the base data for one location is different from that for another site. The location-dependent base data may be sound data related to identifying potential bullying and detection of presence of a moving object. For example, at a bathroom, there are flushing sounds, conversions, cleaning sounds, and etc. Based on the size of the bathroom and the installation location of the sensors 110, the sensors 110 may detect sounds differently from other sensors 110 installed at the bathroom or at a bedroom near the bathroom. Thus, the location-dependent base data may be different based on the installation locations at the same site.

For these reasons, the location-dependent base data is to be obtained at the site for a period in a learning mode. The period may vary depending on the installation location, the time, the day of the week, and the date. The location-dependent base data may be obtained for a period, which is determined based on the environment of the enclosed area 190 and the installation location of the sensors 110. After obtaining location-dependent base data for a period sufficiently long enough to form profile for the location, the sensors 110 may be turned into an active mode to identify noise disturbance.

In an aspect, when the sensors 110 transmits detected results to the control server 120, the control server 120 may acquire from the database 130 the profile for the location where the sensors 110 is installed and the time when the detected results is obtained, and analyzes the detected results to identify occurrence of potential bullying and presence of a moving object based on the base data.

In an aspect, the detected sounds may be used to identify sleep apnea. Sleep apnea is a serious sleep disorder that occurs when a person's breathing is interrupted while sleeping. People with untreated sleep apnea stop breathing repeatedly during their sleep. This means the brain and the rest of the body may not get enough oxygen. Sleep apnea can lead to more serious problems such as high blood pressure, stroke, heart failure, and diabetes.

Similar to potential bullying, base data for sleep apnea may be obtained during the learning mode prior to identifying sleep apnea. During the learning mode, the sensors 110 may record decibel levels of the sleeping sounds of a person over a temporal period, which may be more or less than one week. The base data may contain patterns of the person's breathing at times when the lulls in breathing and loud spikes occur.

In another aspect, the sensors 110 may save the base data in a memory (which is not shown) of the sensors 110. In other words, the sensors 110 may determine vaping, potential bullying, or sleep apnea by itself at the site where the sensors 110 is installed. In this case, the sensors 110 transmits signals indicating abnormality matching signature of vaping, bully, or apnea. This ensures data privacy, meaning that the data stay within the sensors 110, and further ensures privacy of people at the site.

During the active mode, the sensors 110 may listen to the person's sleeping sounds and the control server 120 may compare the current levels (e.g. decibels) of the sleeping with the expected level from the base data at the corresponding time. The comparing data may be displayed so that the user can see when sleep apnea occurs. The control server 120 may measure anomalies in sound over a predicted norm. The control server 120 may determine patterns of snoring, breathing, or any sound disruption during the sleep by analyzing the sound amplitude pattern that occurs. By analyzing the amplitude of the sound as well as irregular levels of sound in the sleep pattern, the control server 120 may identify sleep apnea.

In an aspect, the base data may be location-independent, meaning that the base data is the same for every enclosed location at every time. The location-independent base data may be air quality data related to identifying vaping. Since vaping has a signature in temperature, humidity, and hydrogen ranges, vaping may be identified based on the signature. In an aspect, features for identifying vaping may be integrated into the sensors 110 so that the sensors 110 may request an alert or warning message to be sent to the clients 170, when the signature is identified in the detected air quality or VOCs. The signature may include combination of predetermined ranges of temperature, humidity, and/or hydrogen. Further, predetermined ranges of carbon monoxide, carbon dioxide, nitrogen dioxide, and/or ammonia may also be included in the signature. Furthermore, the signature may be based on total volatile organic compound (TVOC) sensed by one or more VOCs sensors.

Generally, hydrogen sensors require at least 7 volts and about 1,000 ohm resistance. The sensors 110, however, may have a modified hydrogen sensor, which requires much lower voltage and a much higher resistance. The voltage and resistance may vary based on temperature of the environment.

The database 130 may further include history data which is time-series and location-specific data for identifying potential bullying and detection of presence of a moving object for each location where the sensors 110 has been installed. In an aspect, the control server 120 may analyze the history data to predict occurrences of vaping and potential bullying and presence of moving object at the location so that appropriate actions may be proactively and precautiously taken at the location.

In an aspect, the control server 120 may analyze the history data stored at the database 130 to identify trend of the history data. The trend may be a decrease or increase pattern of occurrences of vaping or potential bullying and presence of a moving object. In case a decrease or increase pattern is identified, the control server 120 may adjust the base data for identifying potential bullying and presence of a moving object to make the sensors 110 more or less sensitive to the identification. In this way, the base data may be adjusted based on the trend of the history data.

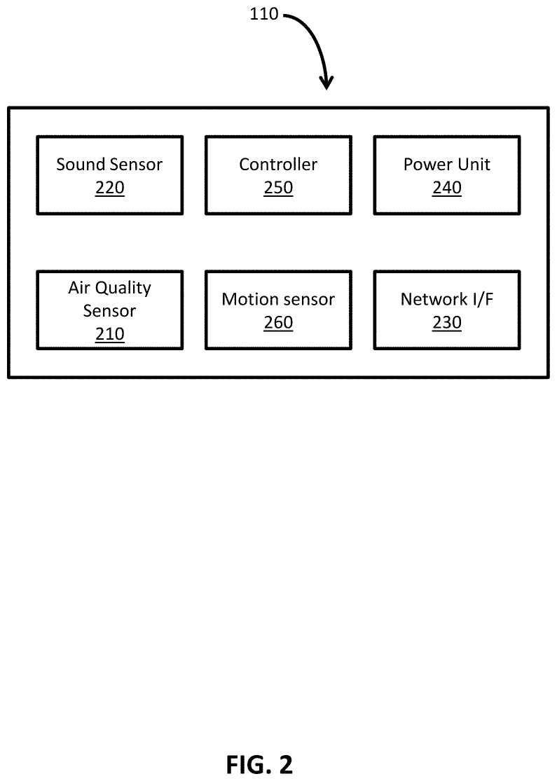

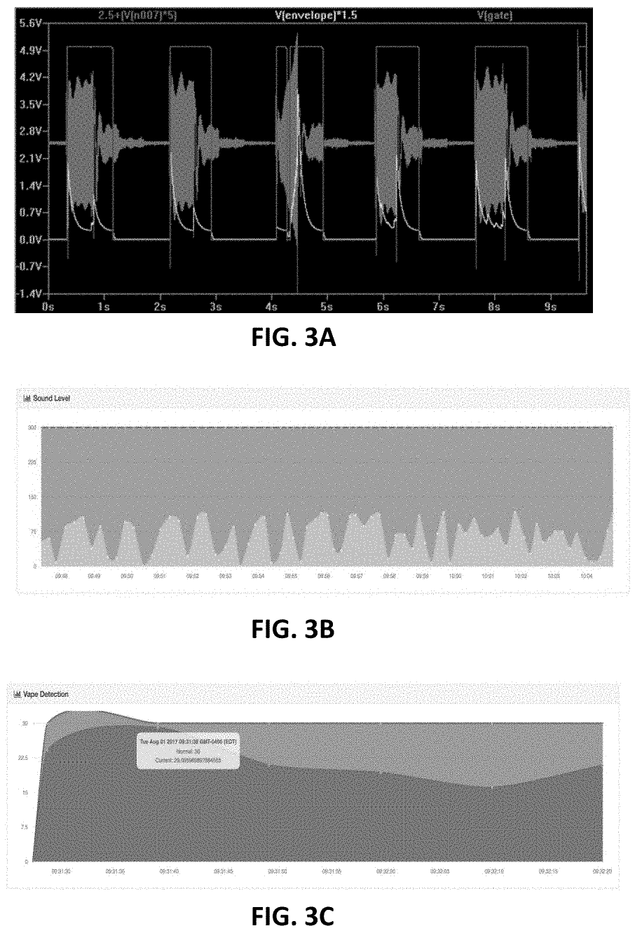

For example, FIGS. 3B and 3C show history data of detected sound level and detected air quality, respectively. The horizontal axes for both graphs of the history data represent time, the vertical axis of FIG. 3B represents decibel or voltage amplitude, and the vertical axis of FIG. 3C represents air quality index. The history data of the detected sounds obtained during the learning mode is used to generate base data for identifying potential bullying, presence of a moving object, or sleep apnea at the installation location in the active mode. As the detected sound fluctuates, the threshold value for identification may vary according to the times. For example, the threshold value for detecting potential bullying at dawn may be lower than the threshold value for detecting potential bullying at noon. It may also vary based on the day of week and location. The threshold value on Wednesday may be higher than on Sunday at a school. On the other hand, the threshold value on Wednesday may be lower than on Sunday at a commercial establishment such as a department store.

Further, the threshold for detecting potential bullying may be higher than a threshold for detection presence of a moving object. In other words, the sound data may be used both in detecting potential bullying and in detecting presence of a moving object.

In an aspect, the sensors 110 may repeat the learning mode and active mode consecutively. As shown in FIG. 3C, the first period (e.g., about ten seconds from the start to 09:31:38) may be used in the learning mode to collect data regarding the environment. Then, the sensors 110 determines whether an adjustment or calibration needs to be made to the modified hydrogen sensor so as to properly detect vaping. For example, the voltage or resistance in the modified hydrogen sensor varies depending on temperature of the environment. Thus, the modified hydrogen sensor can be adjusted or calibrated based on the environment.

After the first period for collecting environment-calibrated data, the threshold value for vaping is determined in the active mode for a second period and the sensors 110 detects vaping based on the threshold value.

In another aspect, the sensors 110 may iterate the learning mode and the active mode after the first and second periods, meaning that the sensors 110 may calibrate the modified hydrogen sensor repeatedly so that the sensors 110 may accurately detect vaping.

FIG. 3C shows two curves. The upper curve represents threshold index value for identifying vaping. The lower curve represents the history data of detection results from the air quality sensor of the sensors 110. The upper curve is stabilized in a period of time after the power-up.

In an aspect, the sensors 110 may repeat the learning mode and active mode consecutively. As shown in FIG. 3C, the first period (e.g., about ten seconds from the start to 09:31:38) may be used in the learning mode to collect data regarding the environment. Then, the sensors 110 determines whether an adjustment or calibration needs to be made to the modified hydrogen sensor so as to properly detect vaping. For example, the voltage or resistance in the modified hydrogen sensor varies depending on temperature of the environment. Thus, the modified hydrogen sensor can be adjusted or calibrated based on the environment.

After the first period for collecting environment-calibrated data, the threshold value for vaping is determined in the active mode for a second period and the sensors 110 detects vaping based on the threshold value.

In another aspect, the sensors 110 may iterate the learning mode and the active mode after the first and second periods, meaning that the sensors 110 may calibrate the modified hydrogen sensor repeatedly so that the sensors 110 may accurately detect vaping based on the index value.

The index value is calculated based on the temperature, moisture, and the detection results of the modified hydrogen sensor. For example, the temperature falls in a range between 60 and 80 degrees Fahrenheit, the moisture is increased by at least 10 percent, and the hydrogen increases from the base level (e.g., environment level) by approximately 10 percent, the sensors 110 may determine that vaping has occurred. This determination is provided as an example and is not provided to limit the scope of this application.

In an aspect, the control server 120 may send a command to the sensors 110 to adjust internal parameters for detecting potential bullying and vaping based on the trend identified from the history data. Further, the control server 120 may communicate with the sensors 110 by calling functions of application programming interface (“API”) between the sensors 110 and the control server 120. In this regard, the sensors 110 can push detection results to the control server 120 and respond to the control server 120's request.

In an aspect, the control server 120 may not store detected results from the sensors 110 because of privacy issues. Nevertheless, the control server 120 may provide signals back to the sensors 110 to indicate tuning parameters and false positives.

Internal parameters of the sensors 110 may include LED functionality, sound threshold, networking server IP address, alert timeout, serial number, reboot for device required or not, latest binary code, vape identification algorithm parameters. This list of parameters should not be understood as exhaustive but provided only for example purposes. The internal parameters of the sensors 110 may further include potential bullying identification algorithm parameters. Potential bullying or vaping identification algorithm parameters may include a window size or threshold values or ranges.

In an aspect, the control server 120 may update internal parameters via text or binary files. Internal parameters for each the sensors 110 may be saved in the database 130.

In another aspect, the control server 120 may control the sensors 110 collectively, individually, or group by group. For example, several the sensors 110 may be installed at the same site. When they need to update internal parameters or settings, the control server 120 may control the sensors 110 collectively at the site. However, such control may not affect the sensors 110 installed in the other sites. The control server 120 may use a query language to request data from the database 130. The query language may be SQL, MySQL, SSP, C, C++, C#, PHP, SAP, Sybase, Java, JavaScript, or any language, which can be used to request data from a database.

In yet another aspect, even when several sensors 110 are installed at the same site, the control server 120 may control them differently because one the sensors 110 may have different parameters for identifying potential bullying and vaping from those of another the sensors 110 due to different installation locations at the site. For example, the sensors 110 installed at a bedroom has parameters different from those of the sensors 110 installed at a bathroom.

Clients 170 may log in to the control server 120 to see graphical representations of the detection results from the sensors 110 via Internet. Communication between the clients 170 and the control server 120 may utilize http, https, ftp, SMTP, or related Internet protocols. The clients 170 may be able to adjust settings for each the sensors 110. For example, the settings may include a mode of warnings (e.g., an email, text message, telephone call, instant message, audible warning, etc.), an address, to which such warnings are to be sent in case of identification of potential bullying or vaping, and the like. The clients 170 are the ones who are responsible for the sites where the sensors 110 are installed for identifying potential bullying and vaping. For example, the clients 170 may be a principal, vice president, or person in charge at a school, a president at a company, a manager at a hospital or any commercial establishment, or security personnel. This list, however, is not meant to be exhaustive but is provided only for showing examples. Other peoples in different rankings, at different locations can be included in this list.

When the sensors 110 identifies potential bullying or vaping, the sensors 110 may send an alert to the clients 170 via a client server 160 using protocols of Internet. The client server 160 may be used for sending a simple message or email to the clients 170 supervising the site, where the potential bullying or vaping is detected. The client server 160 may manage the clients 170 registered on the client server 160 and show alert history and other notification upon requests from the clients 160. Further, the client server 160 may handle customizing or fine tuning the sensors 110, which lead to an alert when the sensors 110 need to reboot, update, or receive configuration. In an aspect, as dotted lines are shown in FIG. 1, the communication between the client server 160 and the clients 170 may not be regularly performed but can be made only when potential bullying or vaping is identified. The clients 170 may receive the alert on a computer, smart device, or mobile phone. In this way, the clients 170 are not swamped by overwhelming number of messages because they receive the alert only when potential bullying or vaping is identified. Further, the clients 170 may be able to timely, properly supervise at the site whenever an alert is received.

When the client server 160 receives an alert from the sensors 110, the client server 160 may communicate with the message server 140, which manages pushing alerts to the notification subscribers 150. The clients 170 may be the persons in charge as the first contact person who has a direct access to the control server 120 for the site, and the notification subscribers 150 may be any related personnel as the second contact persons who do not have a direct access to the control server 120. Similar to the ways the client server 160 sends alerts to the clients 170, the message server 140 sends alerts to the notification subscribers 150 via a text message, email, instant message, telephone call, audible warning, any communication means readily available to a person having skill in the art. The notification subscribers 150 may receive alerts via a computer, smart device, mobile phone, personal digital assistant, tablet, or any available means for receiving such alerts.

As described above, vaping can be identified when the signature is detected, meaning that vaping can be identified independent of locations and times. Thus, features related to identification of vaping may be integrated into the sensors 110. In this case, when vaping is identified, the sensors 110 may bypass the control server 120 and directly communicate with the message server 140 and the client server 160 to transmit alerts to ones in charge or responsible for the sites where the sensors 110 are installed. On the other hand, identification of potential bullying and detection of presence of a moving object is different from site to site due to different environments. In other words, when sounds are detected by the sensors 110, the control server 120 receives and analyzes the detected sounds, and determines whether potential bullying has occurred and whether a moving object is detected. As a result, vaping may be identified earlier than potential bullying, and alerts for vaping may be sent to the notification subscribers 150 and the clients 170 faster than alerts for potential bullying and presence of a moving object.

In an aspect, features for identifying potential bullying and detecting presence of a moving object may be also integrated into the sensors 110. This can be done by the control server 120 controlling the sensors 110 to update internal parameters for identifying potential bullying at the corresponding site. In this case, the control server 120 regularly checks the history data stored at the database 130 and regularly update the internal parameters of the sensors 110 for identifying potential bullying and detecting a presence of the moving object. After updating the internal parameters of the sensors 110, alerts for identifying potential bullying may be sent to the notification subscribers 150 and the clients 170 in the same way as alerts for identifying vaping are sent. When no moving object is detected for a predetermined period, a sterilization measure is performed to sterilize the air in the enclosed area 190.

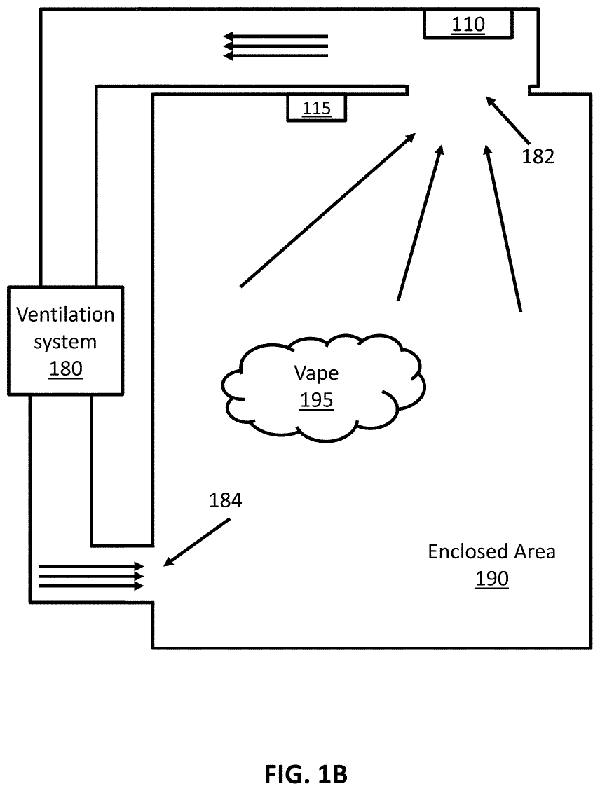

Now referring back to FIG. 2, a functional block diagram of the sensors 110 of FIG. 1 is shown in accordance with embodiments of the present disclosure. The sensors 110 may include a sound sensor 210, an air quality sensor 220, a network interface 230, a power unit 240, a controller 250, and a motion sensor 260. The sound sensor 210 may be used for detecting sound and the air quality sensor 220, such as a VOCs sensor, may be used for detecting air quality.

In particular, the sound sensor 210 detects sound levels (e.g., decibel (dB)) in the environment. For example, FIG. 3A shows detected sound levels in the form of voltage amplitudes. The horizontal axis represents time and the vertical axis represents voltage amplitude. Curves represent detected sound levels in voltage. The bold lines represent windows for identification. For example, the window of identification may be less than 1 second. Within the window, when the voltage amplitude is greater than a threshold value, potential bullying may be identified. In this example, the threshold value is about 4.9 volts. Thus, between 4 and 5 seconds, potential bullying may be identified.

As described above, the threshold values for identifying potential bullying and detecting presence of a moving object depend on the installation location at the site and based on history data obtained during the learning mode. Since the sensors 110 may cover a limited area, several satellite sensors 110 may be installed at one enclosed space when the area of the enclosed space is greater than the area each satellite sensors 110 can cover. For example, the sensors 110 may cover an area of 10 by 10 square feet. In this situation, each satellite sensors 110 may have different threshold value for identifying potential bullying and detecting presence of a moving object due to different installation locations at the same enclosed space.

In an aspect, the threshold value for identifying potential bullying may be great than the threshold value for detecting presence of a moving object.

Also, the presence of a moving object may be detected by the motion sensor 260. In other words, when the motion sensor 260 does not detect any movements for a predetermined time period, it is determined that there is no moving object. When a moving object is not detected for a predetermined period of time by the sound sensor 220 or the motion sensor 260, a sterilizer may sterilize the air at the site. Since the sterilizer may generate a harmful substance, such as ozone, the sterilizer may be activated after the predetermined period of time has passed, i.e., after no movements have been detected for a predetermined period of time.

The air quality sensor 220 may detect air quality including moisture and hydrogen content in the air and temperature of the air. In other words, the air quality sensor 220 may include a combination of sensors sensing air quality. In an aspect, the air quality sensor 220 may include other sensors sensing air content of the environment. Vaping may be detected by specific range combination of humidity, hydrogen, and/or temperature, and/or by detecting VOCs using VOCs sensors/monitors. The detected specific range combination of humidity, hydrogen, and/or temperature, and/or the detected VOCs are identified based on their respective signature. Since the signature does not depend on installation locations and times, internal parameters for identifying vaping may be predetermined. In other words, the air quality sensor 220 does not need training, while the sound sensor 210 needs training. The network interface 230 may be configured to transmit sensed results to the control server 120. In an aspect, the network interface 230 may transmit a request to send an alert, when potential bullying or vaping is identified, to the message server 140 and the client server 160. Further, the network interface 230 may receive a command to update internal settings or parameters from the control server 120.

In an aspect, the network interface 230 may communicate with others wirelessly or via a wired connection. Wireless connections may be wide area network (WAN), local area network (LAN), personal area network (PAN), ad hoc network, cellular network, etc. Wired network may utilize category 5 cable (CAT5), CAT5E, category 6 cable (CAT6), or similar cables. The sound sensor 210, the air quality sensor 220, the network interface 230, and the motion sensor 260 may be powered by the power unit 240. Regular batteries may be installed to supply power to the sensors 110. For example, AA, AAA, or other suitable batteries may be used. The power unit 240 may utilize batteries and a connection to a power outlet so that the power unit 240 may supply power by using the batteries just in case when the power is out.

In an aspect, the power unit 240 may receive power supplied from a network cable, such as CAT5 or CAT6, which is called power-over-Ethernet (PoE) or active Ethernet. PoE+ and 4PPoE may be also used to supply power. Since the network cable supplies power, the sensors 110 may be installed everywhere the network cable can be installed without worrying about a distance to a power outlet. Also, since the power unit 240 does not need electric components necessary for connections to a power outlet, manufacturing cost can be lowered and the size of the sensors 110 can be reduced.

The sensors 110 further includes the controller 250, which controls functions and settings of the sensors 110. When the sensors 110 is powered, the controller 250 sets settings of the sensors 110 and internal parameters of the sound sensor 210 and the air quality sensor 220. The controller 250 further controls the network interface 230 to transmit detected results or requests for sending alerts when potential bullying, sleep apnea, or vaping is detected, and reset or update settings and internal parameters upon reception of update command from the control server 120.

The controller 250 may be implemented on Linux, Windows, android, IOS, or similar software operation system. In an aspect, the controller 250 may be implemented on a hardware system, such as a digital signal processor (DSP), application-specific integrated circuit (ASIC), field-programmable gate array (FPGA), different types of programmable read-only memory (e.g., PROM, EPROM, EEPROM, etc.), or microprocessor such as Raspberry Pi.

In an aspect, the controller 250 may be implemented on a hardware system by removing unnecessary features from the hardware system to reduce power consumption and integrating necessary features for identification into the hardware system. For example, the controller 250 may be implemented on a Raspberry Pi by removing unnecessary features, which were already equipped in the Raspberry Pi, and by integrating features for identifying vaping. In this way, power required for running the sound sensor 210, the air quality sensor 220, the network interface 230, and the controller 250 can be sufficiently supplied via a network cable. This approach for reducing power consumption may be applied to other hardware systems or software operating systems.

In an aspect, the sensors 110 may not be equipped with a warning system. Thus, when potential bullying or vaping is detected at the installation site, any person who bullies or vapes cannot recognize that the identification of such is reported to the clients 170 and the notification subscribers 150 because the identification is reported silently to the person.

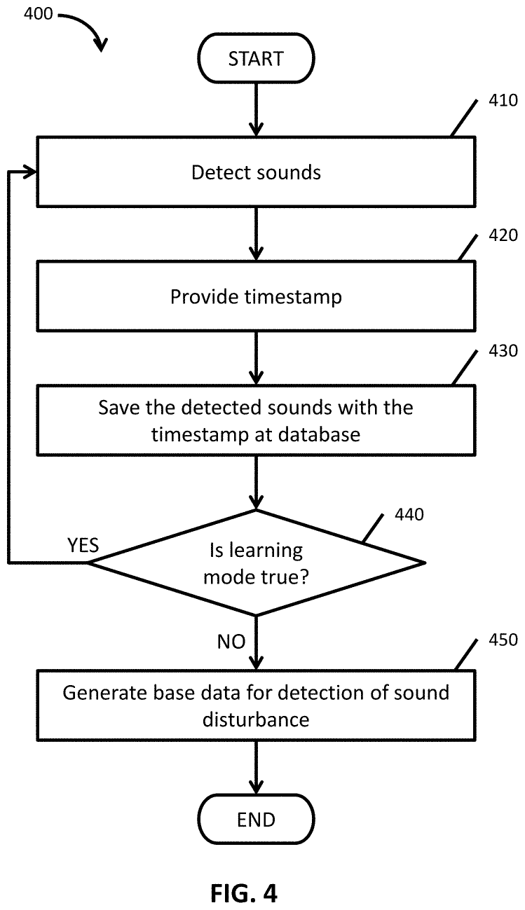

FIG. 4 shows a flowchart for a method 400 in the learning mode in accordance with embodiments of the present disclosure. As described above, the sound sensor 210 of the sensors 110 needs training to generate base data. In the learning mode, the base data is generated. In step 410, the sound sensor detects sounds for a predetermined period. The detected sound is combined with the corresponding timestamp in step 420. The timestamp may include the time, the day of the week, the day, and the month when the sound is detected. The combined data is then saved in a database in step 430.

In step 440, it is checked whether or not the learning mode is still true. If it is true, the method 400 repeats steps 410-440 until sufficient sound data is saved in the database. In an aspect, the sound data may be saved in a memory in the sensors 110 but not in the data base, which is distant from the sensors 110, for protecting privacy.

If it is determined that the learning mode is false in step 440, the method 400 proceeds to step 450, in which base data is generated based on the detected sounds saved at the database during the learning mode. The base data may include a series of threshold values for identifying potential bullying, detecting a presence of a moving object, or sleep apnea along the time of each day, each week, or each month depending on the total duration of the learning mode. After generation of the base data, the method 400 ends.

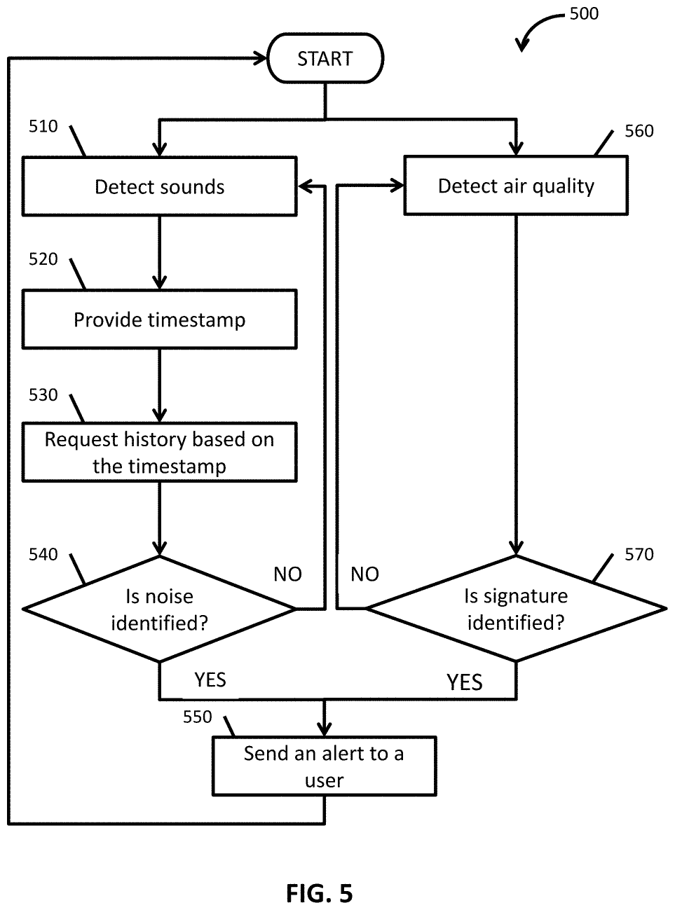

Now turning to FIG. 5, a method 500 is provided in the active mode in accordance with embodiments of the present disclosure. After the base data is generated in method 400 of FIG. 4, the method 500 starts with steps 510 and 560. In step 510, the sound sensor detects sound in the environment and in step 560, the air quality sensor detects air quality. In the method 500, detections of sound and air quality are shown parallelly. In an aspect, such detections may be serially performed.

In step 520, timestamp is provided to the detected sounds. Based on the timestamp, a control system makes a request for history data from the database in step 530. The control system then determines based on the history data whether or not noise disturbance is detected in step 540. The noise disturbance may be related to potential bullying, presence of a moving object, or sleep apnea. In an aspect, the noise disturbance may be related to sound related phenomena or situations, such as fights, hurricane, voice recognition, etc.

If it is determined that the noise disturbance is identified in step 540, the control system silently sends an alert to one or more users who are in charge of the installation site in step 550. After sending the alert, the method 500 restarts the process.

If it is determined that the noise disturbance is not identified in step 540, steps 510-550 are repeated.

Now returning back to the air quality detection, after the air quality is detected in step 560, the control system determines whether or not the signature is identified in step 570. If it is determined that the signature is identified in step 570, the control system silently sends an alert to the one or more users via a text message, email, instant message, optical warning, or oral warning in step 550.

In case when it is determined that the signature is not identified in step 570, the method 500 repeats steps 560 and 570. In this way, sleep apnea, potential bullying, or vaping can be detected and informed to the users. Peoples at the site, however, may not acknowledge the transmission of the alert because the alert is transmitted silently to the people at the site.

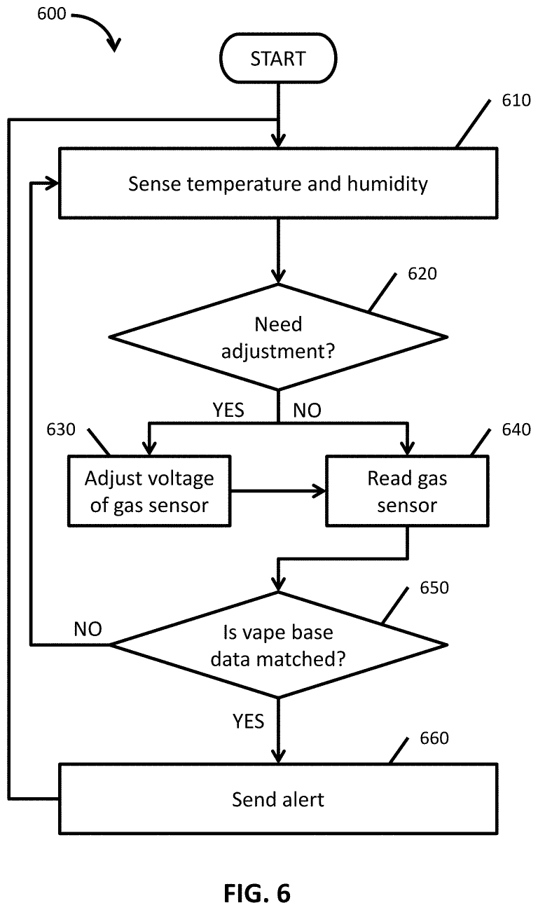

Turning now to FIG. 6, a flowchart is provided for a method 600 for detecting vape. The method starts from sensing temperature and humidity in step 610. As described above, the modified hydrogen sensor of the detection sensor may vary because the voltage or resistance in the modified hydrogen sensor varies depending on temperature of the environment. Thus, in step 620, it is determined whether an adjustment to the modified hydrogen sensor is needed.

When it is determined that the adjustment is needed in step 620, the voltage or resistance of the modified hydrogen sensor is adjusted to appropriately sense gas (e.g., hydrogen) in step 630 and then the method 600 moves to step 640.

When it is determined that the adjustment is not needed in step 620, the modified gas sensor reads gas in step 640.

In step 650, it is determined whether the sensed temperature, humidity, and gas match abnormality matching signature, meaning that the sensed results are within the corresponding ranges. When they match the abnormality matching signature, an alert is sent in step 660. Otherwise, the method 600 goes back to step 610 and repeats steps 610-660.

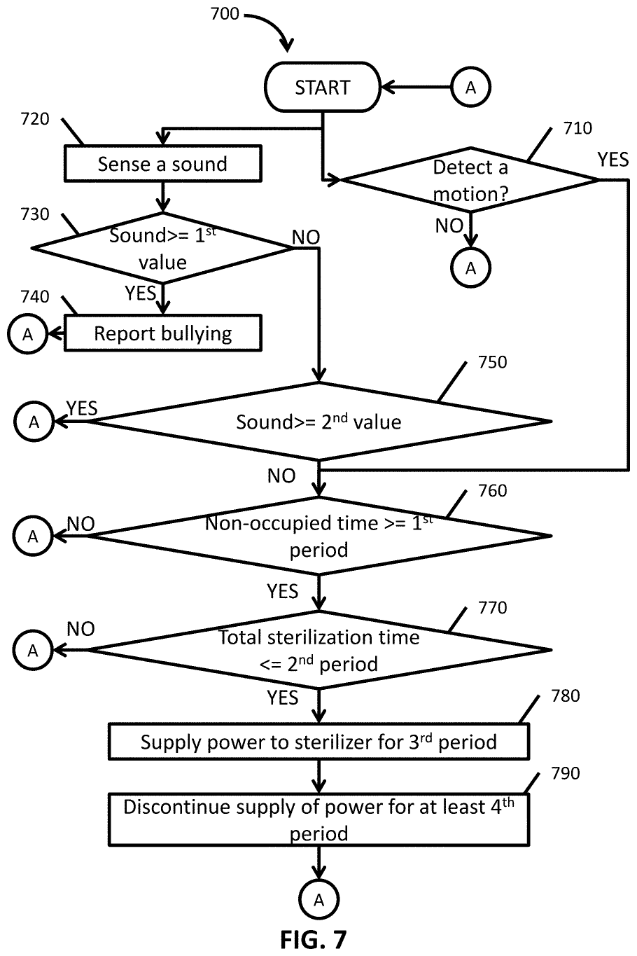

Turning now to FIG. 7, a flowchart is provided for a method 700 for identifying potential bullying and detecting a presence of a moving object. The moving object may be detected when a motion is detected. Thus, the presence of the moving object may be detected in two ways, one by a sound sensor and the other one by a motion sensor.

When a motion is detected by the motion sensor in step 710, the moving object is determined to be present at the enclosed area. Thus, no sterilization of the air is performed and the method 700 keeps checking whether motions are detected.

When sound is detected by the sound sensor in step 720, the detected sound is compared with a first predetermined value for the enclosed area in step 730. The first predetermined value is previously set for identifying potential bullying. Thus, when the detected sound is greater than or equal to the first predetermined value, potential bullying is reported in step 740 and the method 700 keeps checking motions with the sound sensor and the motion sensor.

When it is determined that the detected sound is less than the first predetermined value in step 730, the detected sound is also compared with a second predetermined value for detecting presence of a moving object in step 750. If the detected sound is greater than or equal to the second predetermined value in step 750, that means the moving object is present in the enclosed area. Thus, the method keeps checking the motion and the sound by going back to the starting step.

When it is determined that the detected sound is less than the second predetermined value in step 750, it is determined that there is no presence of a moving object and the method proceeds to step 760, where it is determined whether the continuous non-occupied time is greater than or equal to a first predetermined period. If the continuous non-occupied time is less than or equal to the first predetermined period, then the method keeps checking the sound and motion by going back to the starting step.

When it is determined that the continuous non-occupied time is greater than or equal to the first predetermined period, it is also determined whether or not the total sterilization time is less than or equal to a second predetermined period in step 770. The total sterilization time may be predetermined to ensure minimization of harmful effects of the sterilization. When the total sterilization time is greater than the second predetermined period, the sterilization is done for the unit time (e.g., a day, working hours, business hours, etc.). Thus, no sterilization is performed for the unit time.

When it is determined that the total sterilization time is less than or equal to the second predetermined period in step 770, power is supplied to the sterilizer for a third predetermined period in step 780.

In an aspect, the power may be discontinued during the third predetermined period whenever a moving object is present at the enclosed area. Thus, the actual activation period of the sterilizer may be accumulated to the total sterilization time.

If no moving object is detected during the third predetermined period during activation of the sterilization, supply of the power may be discontinued for at least a fourth predetermined period in step 790. The fourth predetermined period is to ensure that consecutive activation may be temporarily spaced apart and thus the potential harmful substance may be sufficiently dissipated or removed. After step 790, the method 700 keeps checking the presence of the moving object.

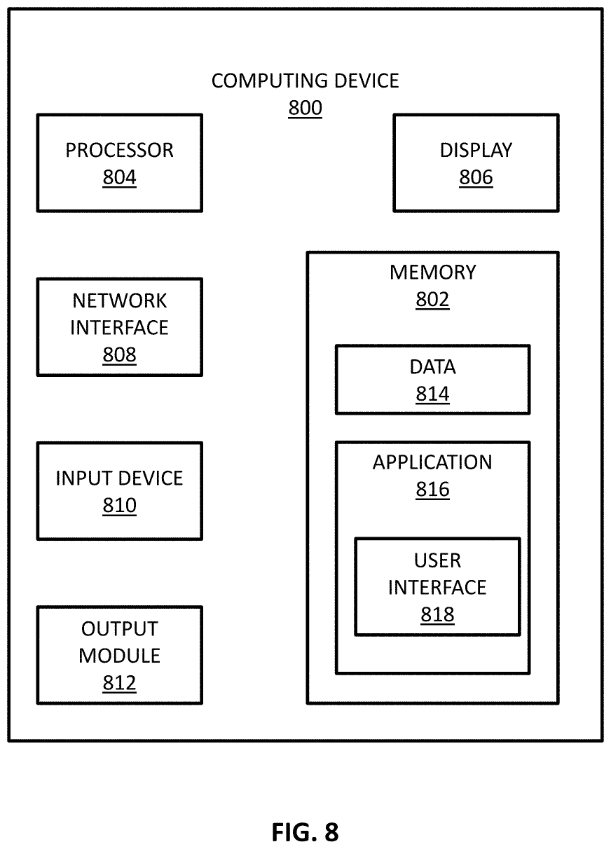

Turning now to FIG. 8, a simplified block diagram is provided for a computing device 800, which can be implemented as the control server 120, the database 130, the message server 140, and the client server 160 of FIG. 1. The computing device 800 may include a memory 802, a processor 804, a display 806, a network interface 808, an input device 810, and/or an output module 812. The memory 802 includes any non-transitory computer-readable storage media for storing data and/or software that is executable by the processor 804 and which controls the operation of the computing device 800.

In an aspect, the memory 802 may include one or more solid-state storage devices such as flash memory chips. Alternatively or in addition to the one or more solid-state storage devices, the memory 802 may include one or more mass storage devices connected to the processor 804 through a mass storage controller (not shown) and a communications bus (not shown). Although the description of computer-readable media contained herein refers to a solid-state storage, it should be appreciated by those skilled in the art that computer-readable storage media can be any available media that can be accessed by the processor 804. That is, computer readable storage media may include non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid state memory technology, CD-ROM, DVD, Blu-Ray or other optical storage, magnetic cassettes, magnetic tape, magnetic disk storage or other magnetic storage devices, or any other medium which can be used to store the desired information and which can be accessed by the computing device 800.

The memory 802 may store application 816 and/or data 814 (e.g., base data and history data from the sound sensor 210 and the air quality sensor 220 of FIG. 2). The application 816 may, when executed by processor 804, cause the display 806 to present the user interface 818 including FIGS. 3A-3C. The processor 804 may be a general purpose processor, a specialized graphics processing unit (GPU) configured to perform specific graphics processing tasks while freeing up the general purpose processor to perform other tasks, and/or any number or combination of such processors. The display 806 may be touch-sensitive and/or voice-activated, enabling the display 806 to serve as both an input and output device. Alternatively, a keyboard (not shown), mouse (not shown), or other data input devices may be employed. The network interface 808 may be configured to connect to a network such as a local area network (LAN) consisting of a wired network and/or a wireless network, a wide area network (WAN), a wireless mobile network, a Bluetooth network, and/or the internet.

For example, the computing device 800 may receive, through the network interface 808, detection results for the sensors 110 of FIG. 1, for example, detected sound in the learning mode and the active mode, and history data, which is time-series data including detected sounds and detected air quality from the sensors 110 for the whole running times or a predetermined period. The computing device 800 may receive updates to its software, for example, the application 816, via the network interface 808. The computing device 800 may also display notifications on the display 806 that a software update is available.

The input device 810 may be any device by means of which a user may interact with the computing device 800, such as, for example, a mouse, keyboard, foot pedal, touch screen, and/or voice interface. The output module 812 may include any connectivity port or bus, such as, for example, parallel ports, serial ports, universal serial busses (USB), or any other similar connectivity port known to those skilled in the art. The application 816 may be one or more software programs stored in the memory 802 and executed by the processor 804 of the computing device 800. The application 816 may be installed directly on the computing device 800 or via the network interface 808. The application 816 may run natively on the computing device 800, as a web-based application, or any other format known to those skilled in the art.

In an aspect, the application 816 will be a single software program having all of the features and functionality described in the present disclosure. In other aspect, the application 816 may be two or more distinct software programs providing various parts of these features and functionality. Various software programs forming part of the application 816 may be enabled to communicate with each other and/or import and export various settings and parameters relating to the identification of potential bullying, sleep apnea, and vaping. The application 816 communicates with a user interface 818 which generates a user interface for presenting visual interactive features to the notification subscribers 150 or the clients 170 of FIG. 1 on the display 806. For example, the user interface 818 may generate a graphical user interface (GUI) and output the GUI to the display 806 to present graphical illustrations such as FIGS. 3A-3C.

Since other modifications and changes may be made to fit particular operating requirements and environments, it is to be understood by one skilled in the art that the present disclosure is not limited to the examples described in the present disclosure and may cover various other changes and modifications which do not depart from the spirit or scope of this disclosure.

What is claimed is:

1. A sensor system for identifying potential bullying and presence of a moving object at a site, the sensor system comprising:
a sound sensor configured to sense sounds at the site;
a motion sensor configured to detect a motion at the site;
a sterilizer configured to sterilize air in the site; and
a controller configured to identify potential bullying based on the sensed sound, identify presence of the moving object at the site based on the sensed sound or detection of the motion, and supply power to the sterilizer based on the identification.

2. The sensor system according to claim 1, wherein the potential bullying is identified when a level of the sensed sound is greater than or equal to a first threshold, and
wherein the presence of the moving object is identified when the level of the sensed sound is greater than or equal to a second threshold, which is less than the first threshold.

3. The sensor system according to claim 1, wherein the sterilizer is an ultraviolet (UV) lamp.

4. The sensor system according to claim 3, wherein the UV lamp emits light having a frequency from about 100 nm to about 280 nm.

5. The sensor system according to claim 3, wherein the UV lamp is turned on for a first predetermined period when the presence of the moving object is identified.

6. The sensor system according to claim 3, wherein the controller is further configured to track a total period during which the UV lamp is powered on.

7. The sensor system according to claim 6, wherein the total period is controlled to be less than or equal to a total predetermined period per unit time based on production of ozone by the UV lamp.

8. The sensor system according to claim 7, wherein the total predetermined period is determined based on an area of the site.

9. The sensor system according to claim 7, wherein the controller is further configured to power off the UV lamp for at least a second predetermined period after the UV lamp is turned on for the first predetermined period.

10. The sensor system according to claim 1, wherein the sterilizer includes hydrogen peroxide.

11. The sensor system according to claim 10, the hydrogen peroxide is sprayed when the sterilizer is powered on.

12. The sensor system according to claim 1, wherein the sensor system is positioned at an entrance of air inflow of a ventilation system.

13. A method for identifying potential bullying and presence of a moving object at a site, the method comprising:
sensing sound by the sound sensor;
detecting a motion of the moving object;
identifying potential bullying from the sensed sound;
identifying presence of a moving object based on the sensed sound or detection of the motion; and
supplying power to a sterilizer after the presence of the moving object is identified.

14. The method according to claim 13, wherein the potential bullying is identified when a level of the sensed sound is greater than or equal to a first threshold, and
wherein the presence of the moving object is identified when the level of the sensed sound is greater than or equal to a second threshold, which is less than the first threshold.

15. The method according to claim 13, wherein supplying the power includes turning on a ultraviolet (UV) lamp for a first predetermined period when the presence of the moving object is identified.

16. The method according to claim 15, further comprising:
tracking a total period during which the UV lamp is powered on.

17. The method according to claim 16, further comprising:
controlling the total period to be less than or equal to a total predetermined period per unit time based on production of ozone by the UV lamp.

18. The method according to claim 17, wherein the total predetermined period is determined based on an area of the site.

19. The method according to claim 17, further comprising:
discontinuing supply of the power to the UV lamp for at least a second predetermined period after the UV lamp is supplied with power for the first predetermined period.

20. A non-transitory computer-readable storage medium including instructions that, when executed by a computer, cause the computer to perform a method for identifying potential bullying and presence of a moving object at a site, the method comprising:
sensing sound by a sound sensor;
detecting a motion of the moving object by a motion sensor;
identifying potential bullying from the sensed sound;
identifying presence of a moving object based on the sensed sound or detection of the motion; and
supplying power to a sterilizer after the presence of the moving object is identified.

21. A sensor system for identifying vaping and detecting presence of a moving object, the sensor system comprising:
an air sensor configured to sense volatile organic compounds (VOCs) in air at a site;
a sound sensor configured to sense sounds at the site;
a motion sensor configured to detect a motion at the site;
a sterilizer configured to sterilize air in the site; and
a controller configured to identify vaping based on signature in the sensed VOCs and identify presence of the moving object at the site based on the sensed sound or detection of the motion, and supply power to the sterilizer based on the identification.

22. The sensor system according to claim 21, wherein the VOCs can include carbon dioxide and carbon monoxide.

23. The sensor system according to claim 21, wherein the signature is predetermined ranges of predetermined parameters.

24. The sensor system according to claim 21, wherein the presence of the moving object is identified when the level of the sensed sound is greater than or equal to a threshold.

25. The sensor system according to claim 21, wherein the sterilizer is an ultraviolet (UV) lamp.

26. The sensor system according to claim 25, wherein the UV lamp emits light having a frequency from about 100 nm to about 280 nm.

27. The sensor system according to claim 25, wherein the UV lamp is turned on for a first predetermined period when the presence of the moving object is identified.

28. The sensor system according to claim 25, wherein the controller is further configured to track a total period during which the UV lamp is powered on.

29. The sensor system according to claim 28, wherein the total period is controlled to be less than or equal to a total predetermined period per unit time based on production of ozone by the UV lamp.

30. The sensor system according to claim 29, wherein the total predetermined period is determined based on an area of the site.

31. The sensor system according to claim 29, wherein the controller is further configured to power off the UV lamp for at least a second predetermined period after the UV lamp is turned on for the first predetermined period.
